(19) United States
(12) Patent Application Publication
(10) Pub. No.: US 2003/0059814 A1
(43) Pub. Date: Mar. 27, 2003

(54) Methods for ribonuclease complementation assays

(76) Inventors: Erik A. Whitehorn, Palo Alto, CA (US); Christopher J. Paddon, Pacifica, CA (US)

Correspondence Address: Edward O. Kreusser, Esq., BROBECK, PHLEGER & HARRISON LLP, 12390 El Camino Real, San Diego, CA, 92130-2081, US

(21) Appl. No.: 10/190,226

(22) Filed: Jul. 3, 2002

Related U.S. Application Data
(60) Provisional application No. 60/303,568, filed on Jul. 5, 2001.

(51) Int. Cl. C12Q 1/68

(52) U.S. Cl. 435/6

(57) ABSTRACT

Methods, compositions, and kits are provided for detecting binding between two members of a specific binding pair. The subject methods employ specific binding members conjugated to complementing RNAse domains to form binding member-complementing domain conjugates. When the specific binding members bind to each other, the members of the complementing RNAse domains are brought into non-covalent contact and reconstitute an active RNAse enzyme by complementation. The subject methods find use in both binding assays and competitive binding assays.

### RELATED APPLICATIONS

This application is related to, and claims priority to, U.S. Provisional Patent Application Serial No. 60/303,568, filed Jul. 5, 2001, entitled Methods for Ribonuclease Complementation Assays, which priority application is herein incorporated by reference in its entirety, including all drawings and sequence listings.

### FIELD OF THE INVENTION

The invention relates to methods of detecting binding between molecules.

### BACKGROUND OF THE INVENTION

There is a present increasing interest and need in being able to assay or monitor a wide variety of organic compounds. Included among compounds of interest are drugs that are employed in the treatment of diseases or aberrant conditions, drugs of abuse, naturally occurring compounds involved with bodily functions, pollutants, trace contaminants and the like. The concentrations of interest of most of these compounds are generally quite small and, in many instances, the environment in which these compounds are found include one or more compounds of similar structure, which must be distinguished from the compound of interest.

For example, pharmaceutical drug discovery, a multi-billion dollar industry, involves the identification and validation of therapeutic targets, as well as the identification and optimization of lead compounds. The explosion in numbers of potential new targets and chemical entities resulting from genomics and combinatorial chemistry approaches over the past few years has placed enormous pressure on screening programs. The rewards for identification of a useful drug are enormous, but the percentage of hits from any screening problem are generally very low.

There is also a need in drug development to determine the ability of a test compound to bind to or inhibit a potential drug target. For example, a large number of drugs are involved with binding to receptor compounds, where a signal may be transduced across membrane, or the effective concentration of the drug is modulated by binding to a receptor, or the binding of a receptor to its ligand may be modulated allosterically. Because it is difficult to define a spatial conformation and charge distribution that is optimal for binding to a particular site, much of drug design involves comparison with a naturally binding substance. It is therefore of great interest to provide a rapid, efficient and inexpensive quantification and screening technique.

### RELEVANT LITERATURE

Characterization, cloning, and sequencing of barnase is discussed by: Hartley and Rogerson (1972) Prep. Biochem. 2:229-242; Hartley et al. (1972) Prep. Biochem. 2:243-250; Hartley and Barker (1972) Nat. New Biol. 235:15-16; Hartley (1976) J. Biol. Chem. 252:3252-3254; and Sancho and Fersh (1992) J. Mol. Biol. 224:741-747.

Complementation assays are described in U.S. Pat. Nos. 4,378,428; 4,708,929; 5,212,064; 5,434,052; 5,604,091; and 5,643,734.

### SUMMARY OF THE INVENTION

Methods, compositions, and kits are provided for detecting binding between members of a specific binding pair. The subject methods employ specific binding members conjugated to complementing RNAse domains to form binding member-complementing domain conjugates. When the specific binding members bind to each other, the members of the complementing RNAse domains are brought into non-covalent contact and reconstitute an active RNAse enzyme that cleaves a labeled RNA probe, e.g. a fluorescent donor quencher pair labeled probe. The subject methods find use in qualitative, quantitative, and competitive binding assays. The assays can be an endpoint or a kinetic assay, and have numerous applications in, for example, drug discovery platforms. In one embodiment of the invention, a binding assay introduces ligands that potentially bridge the two specific binding members, and the presence of a bridging ligand is detected by a readily measurable change in fluorescence intensity. In another embodiment, a candidate ligand is combined with a receptor, and the presence of a binding event is detected by a readily measurable change in fluorescence polarization.

### BRIEF DESCRIPTIONS OF THE DRAWINGS

FIGS. 1A and 1B are graphs depicting barnase cleavage of 5-mer (FIG. 1A) and 33-mer (FIG. 1B) RNA end-labeled with FAM-TAMARA fluorescent donor quencher pair (10′ Barnase (square); 40′ Barnase (triangle); 10′ Control (inverted triangle); 40′ Control (diamond)).

FIG. 2 is a graph depicting cleavage of FAM-TAMARA and FAM-DABCYL quencher-flourescer labeled RNA by dilutions of barnase extracts incubated over time (Barnase (solid); 1:10 Barnase (large checkered); 1:100 Barnase (small checkered); No Extract (cross-hatched).

FIGS. 3A, 3B, and 3C are schematics of IL-5 receptor-barnase fusions IL-5Rα-barnase 1-102, IL-5Rα-barnase 88-110, and IL5Rβ-barnase 88-110, respectively.

FIG. 4 is a graph depicting fluorescence levels after IL-5R barnase fusion complementation in the presence of IL-5Rα dimerizing peptide, AF18748 (no ligand (checkered); 100 nM AF18748 (solid)).

FIG. 5 is a graph depicting the dose-response curve of the peptide AF18748 with IL-5Rα receptor fused to two complementing forms of barnase (10 min (square); 1 hr 17 min (triangle); 6 hr 6 min (inverted triangle)).

FIG. 6 is a graph depicting the use of fluorescence polarization as a readout of enzymatic activity of barnase.

FIG. 7 is a construct employing fusions of the extracellular domain of the erythropoietin receptor to complementing barnase domains.

FIG. 8 is an Epo dose response curve generated using the construct of FIG. 7.

FIG. 9 is a statistical evaluation of the use of the fusion construct of FIG. 7 in a primary screening assay.

### DETAILED DESCRIPTION OF THE INVENTION

Methods, compositions, and kits are provided for high throughput detection of binding between two members of a specific binding pair. The subject methods employ first and second specific binding members conjugated to first and second complementing RNAse domains, respectively, to form first and second binding member-complementing domain conjugates. The binding member-complementing domain conjugates are then combined in an assay medium. When the specific binding members bind to each other, the members of the complementing RNAse domains are brought into non-covalent contact and reconstitute an active RNAse enzyme. The binding between the two members may be direct, or indirectly mediated by a bridging ligand.

The RNAse enzymatic activity is evaluated by a change in signal resulting from cleavage of a labeled RNA provided in the assay medium. In a preferred embodiment, the labeled RNA comprises a fluorescent donor/quencher pair, e.g. FAM and DABCYL. The subject methods find use in assays to determine the ability of a first and second specific binding member to bind to each other, to determine the ability of a bridging ligand to bind to a first and second binding members, and in competitive assays to evaluate the effect of an analyte on the ability of a first and second specific binding member to bind to each other. The methods can be used for quantitative or qualitative assays.

### DEFINITIONS

Before the present methods are described, it is to be understood that this invention is not limited to particular methods described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a specific binding pair” includes a plurality of such specific binding pairs and reference to “the complementing domain” includes reference to one or more complementing domains and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

Specific Binding Member. The term “specific binding member” or “binding member” as used herein refers to a member of a specific binding pair, i.e. two molecules, usually two different molecules, where one of the molecules (i.e., first specific binding member) through chemical or physical means specifically binds to the other molecule (i.e., second specific binding member). The complementary members of a specific binding pair are sometimes referred to as a ligand and receptor; or receptor and counter-receptor. For the purposes of the present invention, the two binding members may be known to associate with each other, for example where an assay is directed at detecting compounds that interfere with the association of a known binding pair. Alternatively, candidate compounds suspected of being a binding partner to a compound of interest may be used.

Specific binding pairs of interest include carbohydrates and lectins; complementary nucleotide sequences; peptide ligands and receptor; effector and receptor molecules; hormones and hormone binding protein; enzyme cofactors and enzymes; enzyme inhibitors and enzymes; etc. The specific binding pairs may include analogs, derivatives and fragments of the original specific binding member. For example, a receptor and ligand pair may include peptide fragments, chemically synthesized peptidomimetics, labeled protein, derivatized protein, etc.

Compounds of interest as binding pair members encompass numerous chemical classes, though typically they are organic molecules. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Compounds are found among biomolecules including peptides, saccharides, fatty acids, steroids, purines, pyrimidines, derivatives, structural analogs or combinations thereof.

Included are pharmacologically active drugs, genetically active molecules, etc. Compounds of interest include chemotherapeutic agents, anti-inflammatory agents, hormones or hormone antagonists, ion channel modifiers, and neuroactive agents. Exemplary of compounds suitable as binding pair members for this invention are those described in The Pharmacological Basis of Therapeutics, Goodman and Gilman, McGraw-Hill, New York, N.Y., (1993) under the sections: Drugs Acting at Synaptic and Neuroeffector Junctional Sites; Drugs Acting on the Central Nervous System; Autacoids: Drug Therapy of Inflammation; Water, Salts and Ions; Drugs Affecting Renal Function and Electrolyte Metabolism; Cardiovascular Drugs; Drugs Affecting Gastrointestinal Function; Drugs Affecting Uterine Motility; Chemotherapy of Parasitic Infections; Chemotherapy of Microbial Diseases; Chemotherapy of Neoplastic Diseases; Drugs Used for Immunosuppression; Drugs Acting on Blood-Forming organs; Hormones and Hormone Antagonists; Vitamins, Dermatology; and Toxicology, all incorporated herein by reference. Also included are toxins, and biological and chemical warfare agents, for example see Somani, S.M. (Ed.), “Chemical Warfare Agents,” Academic Press, New York, 1992).

Analyte. An analyte is any substance whose ability to affect the binding of a first specific binding member to a second specific binding member is to be evaluated. Analytes include drugs and drug metabolites, biologically active molecules, steroids, vitamins, industrial pollutants, pesticides and their metabolites, food additives, herbicides and their metabolites, flavoring agents and food poisons, pathogens and toxins they produce, and any other substances of interest.

Complementing RNAse Domains. By “complementing RNAse domains” or “complementing domains” is meant two enzymatically inactive polypeptides that when non-covalently combined are capable of reconstituting an active RNAse enzyme by the process of complementation. Preferably, the complementing domains have low intrinsic affinity for each other in dilute conditions without specific binding pair-mediated association. The complementing domains should recover sufficient activity when non-covalently combined to provide a detectable difference in the amount of signal as compared to a control in the absence of the complementing domains. Typically, the first and second complementing RNAse domains are both derived from the same RNAse polypeptide.

Suitable complementing domains may be provided by proteolytic cleavage of various RNAse enzymes to provide two fragments. Alternatively, complementing domains can be provided by standard recombinant techniques, where each domain is separately encoded in an expression system.

In one embodiment of the invention, one or both of the RNAse complementing domains are derived from barnase, a small ribonuclease produced extracellularly as a defense mechanism by the bacterium Bacillus amyloliquefaciens (Yazynin and Deyev (1996) FEBS Lett. 388:99-102). Barnase contains 110 amino acids and consists of a single domain formed by two α-helices in the N-terminal region followed by a five-stranded antiparallel β-sheet in the C-terminal region. The first α-helix packs against the β-sheet, thus forming a hydrophobic core that stabilizes the protein. In this embodiment, one complementing domain is derived from the C-terminus of barnase and the other complementing domain is derived from the N-terminus.

Complementation of barnase amino acids 1-102 (barnase 1-102) may be achieved with barnase amino acids 88-110 (barnase 88-110) or 95-110 (barnase 95-110), and complementation of barnase amino acids 1-36 (barnase 1-36) may be achieved with barnase amino acids 37-100 (barnase 37-100). The full sequence of the nucleic acid encoding mature barnase is provided in SEQ ID NO: 1, and the mature barnase polypeptide sequence is provided in SEQ ID NO: 2.

Each of the first and second RNAse complementing domains are chemically linked to one member of a specific binding pair, i.e. a first complementing domain is linked to a first specific binding member and a second complementing domain is linked to a second specific binding member, such that when the first and second binding members bind to each other, the first and second complementing domains are brought into sufficient proximity that an active RNAse is reconstituted. In referring to these conjugates, the terms “first binding member-complementing domain conjugate” or “first conjugate”, and “second binding member-complementing domain conjugate” or “second conjugate” may be used. Those of skill in the art will appreciate that the designation “first” and “second” is merely a matter of convenience.

A large number of linking groups may be employed for joining a wide variety of specific binding members to a functionality present in a complementing domain. Chemical groups that find use in linkage include carbamate; amide (amine plus carboxylic acid); ester (alcohol plus carboxylic acid), thioether (haloalkane plus sulfhydryl; maleimide plus sulfhydryl), Schiff's base (amine plus aldehyde), urea (amine plus isocyanate), thiourea (amine plus isothiocyanate), sulfonamide (amine plus sulfonyl chloride), disulfide; hydrazone, lipids, and the like, as known in the art. Ester and disulfide linkages are preferred if the linkage is to be readily degraded in the cytosol after transport of the substance. Various functional groups (hydroxyl, amino, halogen, etc.) can be used to attach the cargo or targeting domain to the transport domain. The linkage may also comprise spacers, e.g. alkyl spacers, which may be linear or branched, usually linear, and may include one or more unsaturated bonds; usually having from one to about 300 carbon atoms; more usually from about one to 25 carbon atoms; and may be from about three to 12 carbon atoms. Spacers of this type may also comprise heteroatoms or functional groups, including amines, ethers, phosphodiesters, and the like.

The linkage may be a homo- or heterobifunctional linker having a group at one end capable of forming a stable linkage to the transport domain, and a group at the opposite end capable of forming a stable linkage to the cargo. Illustrative entities include: azidobenzoyl hydrazide, N-[4-(p-azidosalicylamino)butyl]-3′-[2′-pyridyldithio]propionamide), bis-sulfosuccinimidyl suberate, dimethyladipimidate, disuccinimidyltartrate, N-γ-maleimidobutyryloxysuccinimide ester, N-hydroxy sulfosuccinimidyl-4-azidobenzoate, N-succinimidyl [4-azidophenyl]-1,3′-dithiopropionate, N-succinimidyl [4-iodoacetyl]aminobenzoate, glutaraldehyde, NHS-PEG-MAL; succinimidyl 4-[N-maleimidomethyl]cyclohexane-1-carboxylate; 3-(2-pyridyldithio) propionic acid N-hydroxysuccinimide ester (SPDP); N, N′-(1,3-phenylene) bismaleimide; N, N′-ethylene-bis-(iodoacetamide); or 4-(N-maleimidomethyl)-cyclohexane-1-carboxylic acid N-hydroxysuccinimide ester (SMCC); m-maleimidobenzoyl-N-hydroxysuccinimide ester (MBS), and succinimide 4-(p-maleimidophenyl)butyrate (SMPB), an extended chain analog of MBS. The succinimidyl group of these cross-linkers reacts with a primary amine, and the thiol-reactive maleimide forms a covalent bond with the thiol of a cysteine residue.

Other cross-linking reagents useful for this purpose include: p,p′-difluoro-m,m′-dinitrodiphenylsulfone (which forms irreversible cross-linkages with amino and phenolic groups); dimethyl adipimidate (which is specific for amino groups); phenol-I, 4-disulfonylchloride (which reacts principally with amino groups); hexamethylenediisocyanate or diisothiocyanate, or azophenyl-p-diisocyanate (which reacts principally with amino groups); bisdiazobenzidine (which reacts primarily with tyrosine and histidine); O-benzotriazolyloxy tetramethuluronium hexafluorophosphate (HATU), dicyclohexyl carbodiimde, bromo-tris (pyrrolidino) phosphonium bromide (PyBroP); N,N-dimethylamino pyridine (DMAP); 4-pyrrolidino pyridine; N-hydroxy benzotriazole; and the like.

Homobifunctional cross-linking reagents include bismaleimidohexane (“BMH”). BMH contains two maleimide functional groups, which react specifically with sulfhydryl-containing compounds under mild conditions (pH 6.5-7.7). The two maleimide groups are connected by a hydrocarbon chain, and are useful for linking polypeptides that contain cysteine residues.

Where a specific binding member is a polypeptide, a binding member-complementing domain conjugate can be created using a fusion protein which may be made by ligating or fusing a gene encoding the complementing domain with another gene encoding the binding member, or a portion thereof. The expression of the ligated genes in an appropriate host cell results in a fusion protein product that is capable both of complementation with- a second complementing domain and specific binding to a second specific binding member. Thus, fusion proteins prepared according to this embodiment of the present invention comprise two domains: (1) a complementing domain, and (2) a specific binding member domain, both encoded by a fused gene.

To construct a gene that encodes a binding member-complementing domain fusion protein, the two genes in question must be joined with their coding sequences such that the translational reading frame is maintained and is uninterrupted by termination signals. Further, if the host cell is a strain that contains a repressor, the fusion protein will be produced only in response to inactivation of the repressor of induction. The host cell can then be screened for expression of the fusion protein. Fusion proteins can be constructed where the specific binding member is attached to either the N-terminus or C-terminus of the complementing domain. A spacer sequence between the complementing domain and the binding member can be used to enhance complementation.

Labeled probe. An cleavable ribonucleic acid molecule, comprising a detectable label. The molecule may be an RNA molecule, or may be a molecule comprising an RNA linkage, e.g. PNA, nucleic acid analogs, DNA, etc., where one or more of the nucleoside linkages is cleavable with RNAse. Detectable labels include isotopic labels, in which one or more of the nucleotides is labeled with a radioactive label, such as ³⁵S, ³²p, ³H, etc. Fluorescent labels of interest include: fluorescein, rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), the cyanine dyes, such as Cy3, Cy5, Alexa 542, Bodipy 630/650, fluorescent particles, fluorescent semiconductor nanocrystals, and the like.

In one embodiment of the invention, the label is detected by release of the labeled moiety from the probe, for example where the probe is bound to a solid substrate. In another embodiment, the label is undetectable until cleavage, e.g. in a donor/quencher pair, and the reaction vessel is monitored for a change in fluorescence resulting from cleavage.

The labeled probe can comprise an end bound to a solid substrate and a free end, or may comprise two free ends. Where the labeled probe is bound to a solid support, the label is typically attached to the probe at or near the free end. Where the probe is not attached to a solid support, the label may be attached at any point along the length of the probe, but is typically attached at or near one end.

In one embodiment, the probe is a self-quenching fluorescence probe comprising a reporter dye and a quencher dye (donor-quencher pair). Upon RNAse cleavage of the ribonucleotides in the probe, the fluorescer and quencher separate so that a fluorescent signal is detectable. The self-quenching probe is designed so as to bring the reporter into close proximity with the quencher, which permits efficient energy transfer from the reporter to the quencher. A donor-quencher pair comprises two fluorophores having overlapping spectra, where the donor emission overlaps the acceptor absorption, so that there is energy transfer from the excited fluorophore to the other member of the pair. It is not essential that the excited fluorophore actually fluoresce, it being sufficient that the excited fluorophore be able to efficiently absorb the excitation energy and efficiently transfer it to the emitting fluorophore.

The donor fluorophore is excited efficiently by a single light source of narrow bandwidth, particularly a laser source. The emitting or accepting fluorophores will be selected to be able to receive the energy from the donor fluorophore and emit light. Usually the donor fluorophores will absorb in the range of about 350-800 nm, more usually in the range of about 350-600 nm or 500-750 nm, while the acceptor fluorophores will emit light in the range of about 450-1000 nm, usually in the range of about 450-800 nm.

The two fluorophores will be joined by an RNAse cleavable bond, which may be provided as an RNA polynucleotide, where the distance between the two fluorophores may be varied. The transfer of the optical excitation from the donor to the acceptor depends on the distance between the two fluorophores. Thus, the distance must be chosen to provide efficient energy transfer from the donor to the acceptor. Various conventional chemistries may be employed to ensure that the appropriate spacing between the two fluorophores is obtained. The fluorophores may be bound internal to the chain, at the termini, or one at one terminus and another at an internal site.

The fluorophores may be selected so as to be from a similar chemical family or a different one, such as cyanine dyes, xanthenes or the like. Reporter, or donor, dyes of interest include: fluorescein dyes (e.g., 5-carboxyfluorescein (5-FAM), 6-carboxyfluorescein (6-FAM), 2,4′,1,4,-tetrachlorofluorescein (TET), 2′,4′,5′,7′,1,4-hexachlorofluorescéin (HEX), and 2′,7′-dimethoxy-4′,5′-dichloro-6-carboxyfluorescein (JOE)), cyanine dyes, dansyl derivatives, and the like. Quencher dyes of interest include: rhodamine dyes (e.g., tetramethyl-6-carboxyrhodamine (TAMRA), and tetrapropano-6-carboxyrhodamine (ROX)), DABSYL, DABCYL, cyanine, anthraquinone, nitrothiazole, and nitroimidazole compounds, and the like. The labeled RNA probe can be produced using any convenient protocol.

Cleavage of the RNA by the active RNAse enzyme results in a change of detectable signal, which may be disappearance or appearance of label, e.g. fluorescence. Where the labeled probe is bound to a solid support, cleavage of the RNA allows unbound label to be washed and removed from the substrate. Where the probe is a self-quenching fluorescence probe, cleavage of the RNA allows the fluorescer and quencher to separate so that fluorescence is detectable.

In another embodiment, detection of the RNAse cleaved labeled probe can be accomplished using fluorescence polarization, a technique to differentiate between large and small molecules based on molecular tumbling. When fluorescent molecules are excited with plane polarized light, they emit light in the same polarized plane, provided that the molecule remains stationary throughout the excited state. However, if the excited molecule rotates or tumbles out of the plane of polarized light during the excited state, then light is emitted in a different plane from that of the initial excitation. For example, if vertically polarized light is used to excite the fluorophore, the emission light intensity can be monitored in both the original vertical plane and also the horizontal plane. The degree to which the emission intensity moves from the vertical to horizontal plane is related to the mobility of the fluorescently labeled molecule. If fluorescently labeled molecules are very large (e.g., intact labeled probe), they move very little during the excited state interval, and the emitted light remains highly polarized with respect to the excitation plane. If fluorescently labeled molecules are small (e.g., cleaved labeled probe), they rotate or tumble faster, and the resulting emitted light is depolarized relative to the excitation plane. Thus, upon linkage of a fluorescent moiety to the ribonucleotides of the probe, the presence of the fluorescent moiety can be differentiated based on molecular tumbling, thus differentiating between intact and RNAse cleaved probe.

By “solid substrate” or solid support” is meant any surface to which the labeled probes of the subject invention are attached. A variety of solid supports or substrates are suitable for the purposes of the invention, including both flexible and rigid substrates. By flexible is meant that the support is capable of being bent, folded or similarly manipulated without breakage. Examples of flexible solid supports include nylon, nitrocellulose, polypropylene, polyester films, such as polyethylene terephthalate, etc. Rigid supports do not readily bend, and include glass, fused silica, quartz, acrylamide; plastics, e.g. polytetrafluoroethylene, polypropylene, polystyrene, polycarbonate, and blends thereof, and the like; metals, e.g. gold, platinum, silver, and the like; etc. The substrates can take a variety of configurations, including planar surfaces, filters, fibers, membranes, beads, particles, dipsticks, sheets, rods, etc.

In one embodiment of the invention, the substrate comprises a planar surface, and labeled probes are attached to the surface. The probes may be attached in a uniform pattern or in an array in a plurality of probe spots. The density of labeled probes on the substrate will be such that a signal from the label can be detected and the reconstituted RNAse can cleave the RNA of the probe. As such, the density will vary depending on the identity of the particular label and RNAse employed. Where the probes are spotted on the array, the spots can be arranged in any convenient pattern across or over the surface of the support, such as in rows and columns so as to form a grid, in a circular pattern, and the like, where generally the pattern of spots will be present in the form of a grid across the surface of the solid support. The total number of probe spots on the substrate will vary depending on the concentration of binding member-complementing domain conjugates, as well as the number of control spots, calibrating spots and the like, as may be desired.

In another embodiment, the substrate is a collection of physically discrete solid substrates, e.g. a collection of beads, individual strands of fiber optic cable, and the like. Each discrete substrate can have probes distributed across the surface or attached in one or more probe spots on the substrate. The collection of physically separable discrete substrates may be arranged in a predetermined pattern or may be separated in a series of physically discrete containers (e.g., wells of a multi-well plate).

The subject substrates can be prepared using any convenient means. One means of preparing the supports is to synthesize the probes, and then deposit on the support surface. The probes can be deposited on the support using any convenient methodology, including manual techniques, e.g. by micropipette, ink jet, pins, etc., and automated protocols. Of particular interest is the use of an automated spotting device, such as the Beckman Biomek 2000 (Beckman Instruments). Alternatively, the probes can be synthesized on the substrate using standard techniques known in the art.

In an alternative method, where the probes are not bound to a solid support, the assays of the invention may utilize such reaction vessels as 96 well plates, etc., as are known in the art.

### Assay Methods

A wide variety of protocols can be used in conjunction with the present invention. In one embodiment, the assay is designed to evaluate the ability of two members of a specific binding pair to bind to each other. In this embodiment, a first and second binding member-complementing domain conjugate are combined in assay medium. Binding of the specific binding members of the binding member-complementing domain conjugates brings the members of the complementing RNAse domains into non-covalent contact and an active RNAse enzyme is reconstituted. Thus, enzyme activity directly indicates the extent of interaction between the first and second specific binding members.

The labeled probe may be added at the same time as the conjugates or after an incubation period. Where the labeled probe is bound to a solid support, after RNAse cleavage the support is washed of unbound probe before label is detected. RNAse cleavage of the labeled probe can be evaluated at a single or multiple time points, or can be monitored continuously. A change in signal, e.g. a change in amount of fluorescence, indicates RNAse cleavage of the labeled probe, which in turn indicates binding of the specific binding members.

In another embodiment, the subject methods can be used to evaluate the effect of an analyte on the ability of members of a specific binding pair to bind to each other. In this embodiment, a first and second binding member-complementing domain conjugate are combined in assay medium along with an analyte of interest. Binding of the specific binding members of the conjugates brings the members of the complementing RNAse domains into non-covalent contact and an active RNAse enzyme is reconstituted. When compared to a control without analyte, RNAse activity will be changed if the analyte affects binding between the specific binding members. Thus, enzyme activity indicates the affect of the analyte on interaction between the first and second specific binding members. Enzymatic activity can be determined by comparing the signal obtained after adding the first and second conjugates to the signal before adding the conjugates, or to a control.

These methods find use in determining the ability of an analyte to inhibit or promote binding between members of a specific binding pair, determining the ability of an analyte to bind to a specific binding member, quantifying an analyte, and the like. In this embodiment, either a competitive or equilibrium mode can be employed. In a competitive mode, both the analyte and a first specific binding member compete for binding to a second specific binding member. In an equilibrium mode, the analyte is allowed to interact with the second specific binding member for a sufficient time to approach equilibrium, after which time the first specific binding member is added. The first specific binding member can then only bind with second specific binding member that it is not sterically hindered from binding by the presence of the analyte.

In the competitive protocol, the analyte and first conjugate may be added concomitantly to the assay medium containing the second conjugate and the labeled probe. The signal can be determined at a single or multiple time points, or can be monitored continuously, after addition of the reagents to the assay medium and compared to the signal before addition of the reagents or to a control signal. The difference in these two values indicates enzymatic activity. The signals can also be compared to values obtained with known amounts of the analyte to determine the amount of analyte present. Alternatively, the labeled probe and second conjugate can be added to the assay medium after addition of the analyte and first conjugate. Various incubation times can be employed between the addition of the reagents and the signal measurement.

In the equilibrium mode, the analyte can be added to the second conjugate concomitantly with the first conjugate or followed by the addition of the first conjugate. In one embodiment of this mode, after addition of the analyte and second conjugate, the assay medium may be incubated for a sufficient time to approach equilibrium, followed by addition of the first conjugate. The medium may then be incubated a second time followed by signal measurements for enzymatic activity. Alternatively, the analyte can be added to the assay medium and incubated, followed by the addition of the second conjugate and a further incubation, followed by the addition of the first conjugate and optionally a third incubation. The incubation times will vary widely, and may be less than about 0.5 minute and usually not exceeding 24 hours, more usually not exceeding 6 hours, and preferably not exceeding about 30 minutes. Where the ultimate result will be dependent upon the results obtained with standard(s) treated in substantially the same manner and when possible in the identical manner, the particular mode and periods of time are not critical, so long as significant reproducible differentiations are obtained with varying concentrations of analyte.

While one signal measurement may suffice, it is preferable to take two spaced apart measurements for each assay and report the results as the difference between the two values. Alternatively, the signal may be compared to a control. In both competitive and equilibrium modes, where the labeled probe is bound to a solid support, after RNAse cleavage the support is washed of unbound probe before signal is detected. The skilled artisan will recognize that order of addition of reagents and timing of signal measurements can be varied and need not exactly follow those described above.

The concentration of analyte that may be assayed will generally vary from about 10⁻⁴ to 10⁻¹⁵ M, more usually from about 10⁻⁶ to 10⁻¹³ M. One of skill in the art can readily determine the relative amounts of each reagent based on considerations such as whether the assay is qualitative, semiquantitative or quantitative, the particular RNAse and method of detection of enzymatic activity. Where an analyte is present, the concentration of the analyte of interest will normally determine the concentration of the other reagents. Typically, labeled probe will be present in excess such that it does not limit detection of enzymatic activity, etc. Additional guidance for optimizing assay conditions can be found in the literature, e.g. U.S. Pat. Nos. 4,233,401 and 4,373,428, the disclosures of which are incorporated herein by reference, Principles of Competitive Binding Assays, Odell ed (1983) John Wiley and Sons, Inc., New York, N.Y. (particularly at pages 141-147), and Eruk et al. (1984) Ann. Clin. Biochem. 21:434-443.

In general the assays are carried out in an aqueous buffered medium at a moderate pH and the assay reagents can be added successively or concomitantly. The assay medium can include other polar solvents, such as oxygenated organic solvents of from 1 to 6, more usually of from 1 to 4 carbon atoms, including alcohols, ethers and the like. Usually these cosolvents will be present in less than about 20 weight percent, more usually in less than about 10 weight percent. The subject assays can be carried out in any convenient container, such as test tubes, microfuge tubes, microtiter plate wells, and the like, and can be carried out in parallel in different containers (e.g., different test tubes) or in different compartments of a single container (e.g., different wells in a multi-well microtiter plate).

The pH for the medium will usually be in the range of from about 5 to 10, more usually in the range of from about 7 to 9 and preferably from about 7 to 8.5. Various buffers may be used to achieve the desired pH and maintain the pH during the determination. Illustrative buffers include borate, phosphate, carbonate, tris, barbital, and the like. The particular buffer employed is not critical to this invention, but in individual assays one buffer may be preferred over another.

Moderate temperatures are normally employed for carrying out the assay and usually constant temperatures during the period of the assay measurement will be employed. The temperatures will normally be in the range of from about 100 to 50° C., more usually from about 15° to 40° C.

### Kits

Also provided by the subject invention are kits for detecting binding between two members of a specific binding pair. The kits may comprise containers, each with one or more of the various reagents used in the subject methods, including, for example, a first complementing RNAse domain, a second complementing RNAse domain, a first specific binding member, a second specific binding member, a labeled probe (either free or bound to a solid support), analytes, assay containers, assay medium, buffer, etc. In addition, the kits can also comprise a first binding member-complementing domain conjugate and a second binding member-complementing domain conjugate. The kits can further comprise a set of instructions, where the instructions may be associated with a package insert and/or the packaging of the kit or kit components.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Barnase Cleavage of End-labeled Fluorescent RNA

To test the ability of barnase to cleave end-labeled fluorescent RNA, an E. coli expressed barnase was used with a FAM-TAMARA fluorescent donor-quencher pair labeled 5-mer and 33-mer RNAs (5′-FAM-gcgaa-TAMARA-3′ and 5′-FAM-ccggccgccgcuuuuuuuuuuugcggcggccgg-TAMARA-3′ (SEQ ID NOS: 3 and 4, respectively)), and with a FAM-DABCYL fluorescent donor-quencher pair labeled 5-mer RNA (5′-FAM-gcgaa-DABCYL-3′).

Briefly, 50 ml cultures of DH5α (“Control”) or the bacterial strain ARI# 1890 (“Barnase”) containing the barnase (Bacillus amyloliquefaciens extracellular ribonuclease) expression plasmid (from Robert W. Hartley, NIH) in the same host bacterium, DH5α, were inoculated from a single colony and grown at 37 degrees for 24 hours. Cells were centrifuged and then suspended in 30 mM Tris-HCI/20% sucrose, pH 8.0 and EDTA added to 1 mM for 10 minutes at room temperature. Cells were centrifuged again and the pellet suspended in ice-cold 5 mM MgSO₄. This suspension was then centrifuged again and the supernatant saved as the “Osmotic Shock Prep”. Dilutions of this material were performed in TE buffer with 1 μM RNA and fluorescence measured after 10′ or 40′ in the Tecan instrument using the fluorescein settings.

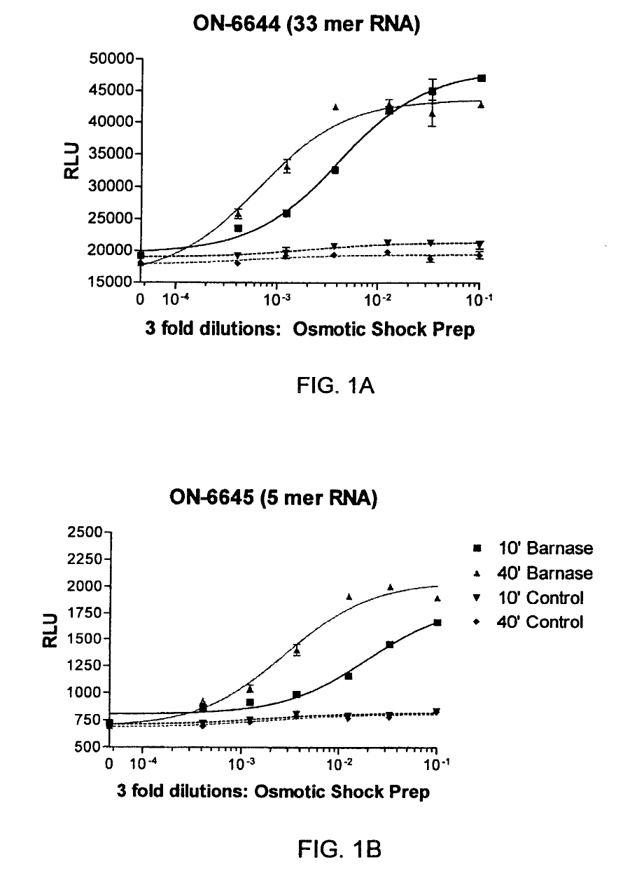

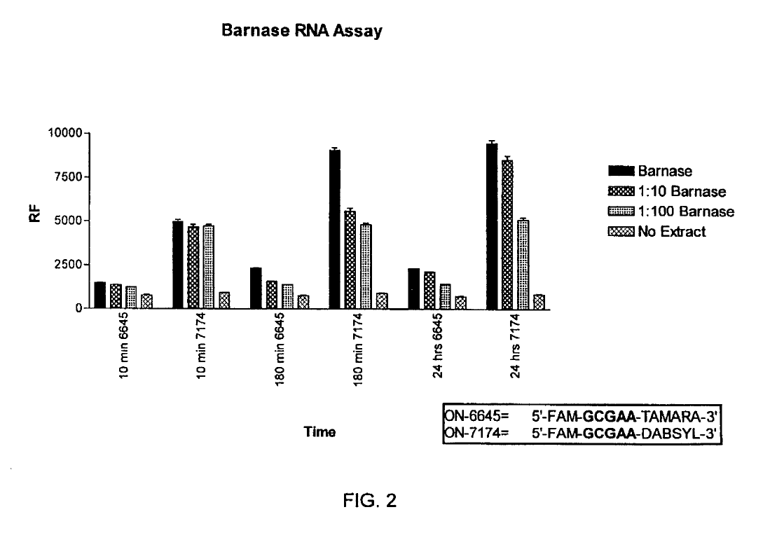

The results demonstrated the expected increases in fluorescence in the homogeneous assay after barnase cleavage of the FAM-TAMRA 5-mer and 33-mer RNAs (FIGS. 1A and 1B). In addition, the 5-mer model sequence, FAM-TAMARA conjugate was compared to that of the FAM-DABCYL pair (FIG. 2). These results indicated superior signals could be obtained with the FAM-DABCYL pair.

### Example 2

### In Vitro Barnase Complementation

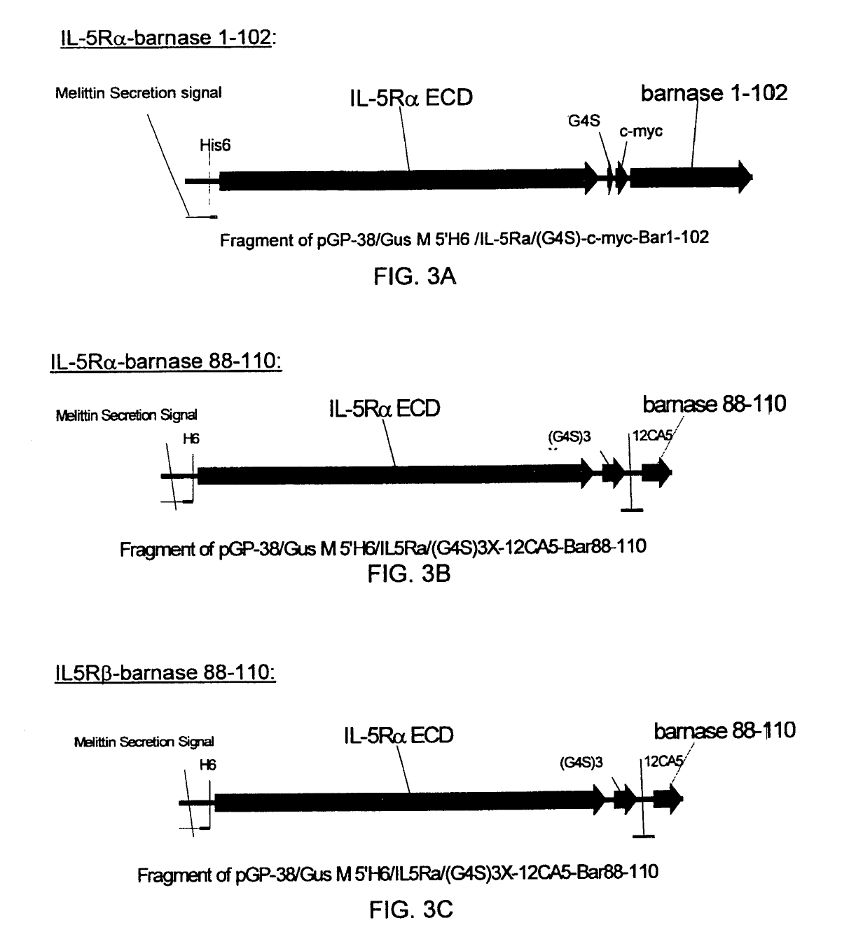

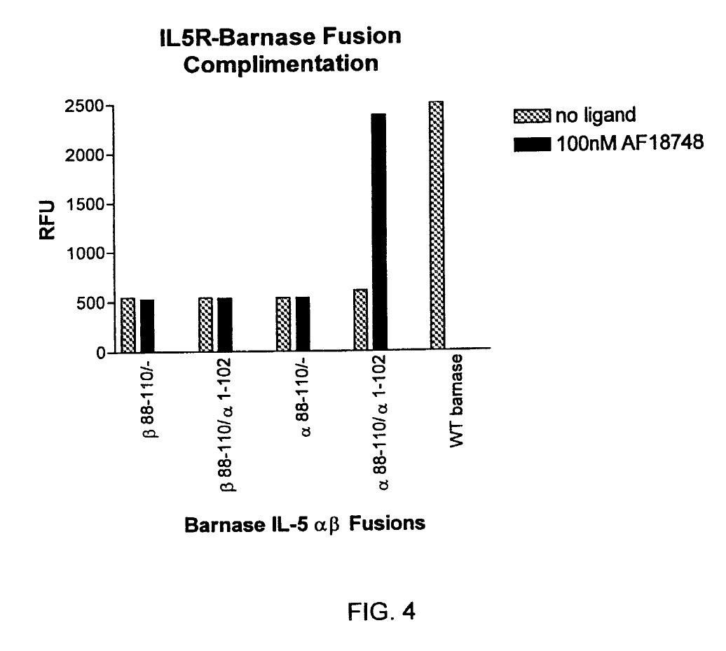

To test the in vitro potential for ligand-mediated barnase complementation, fusions to the extracellular domains of the α and β chains of the IL-5 receptor were constructed (FIGS. 3A, 3B, and 3C), expressed in baculovirus infected insect cells and purified. Purified IL-5α and β receptor barnase fusions were then prepared by Agarose Ni-NTA chromatography. A liquots of approximately equal concentration of differing combinations of these proteins were incubated in 80 mM imidazole/phosphate buffer, pH 8.0 with the 5-mer RNA, 5′-FAM-gcgaa-DABCYL-3′, in the presence or absence of the peptide AF18748 (England et al., (2000) P.N.A.S. 97:6862-6867). The fluorescence intensity (530 nm) was then measured on the Tecan instrument after 3 hours. The results of testing the IL-5Rα dimerizing peptide, AF18748 are shown in FIG. 4. The peptide has no effect on the α alone or β alone proteins or upon the α/β mixture. As predicted, only the α/α complementing pair induce an increase in the fluorescent signal.

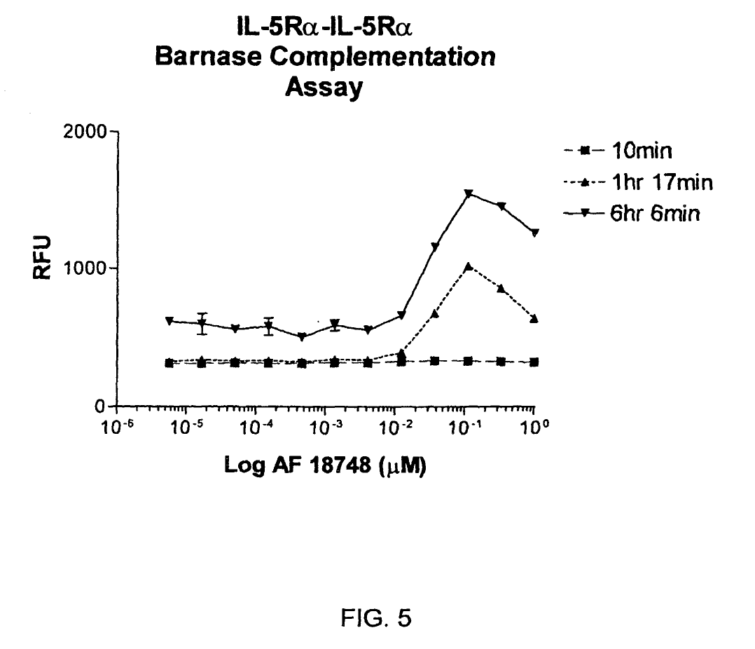

Subsequently, a dose response curve was determined for the AF1 8748 peptide (FIG. 5). The result was equivalent to the bell shaped dose-response curve obtained by the FMAT technique (England et al.). The above results indicate that the presence of the barnase complementing domains did not interfere with AF1 8748-mediated dimerization of IL-5Rα and that complementation reconstitutes a functional barnase.

### Example 3

### Fluorescence Polarization as a Readout of Enzymatic Activity

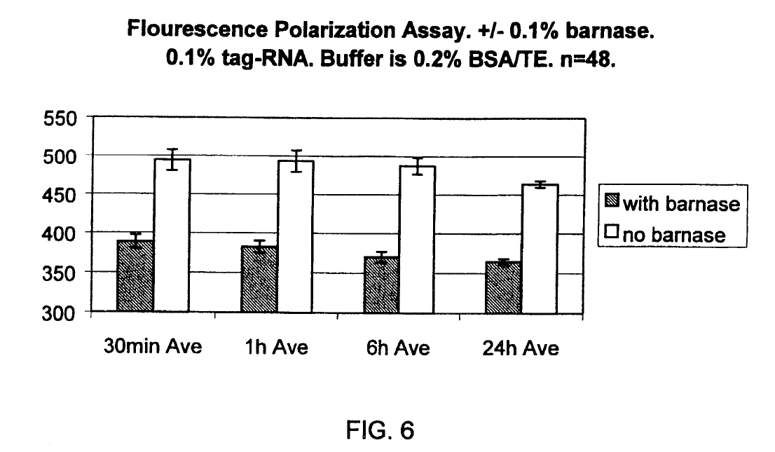

To examine the use of fluorescence polarization as a readout for enzymatic activity, an 18 and 28 S RNA preparation (Sigma Chemical Co.) was labeled with fluorescein using the VersaTag labeling kit (NEN Life Science Products, Inc.) according to the manufacturer's directions. The labeled RNA was incubated with barnase in a 96-well plate for 30 min, 1 hour, 6 hours, or 24 hours. After incubation, the change in fluorescence polarization (mP) was measured with a LJL BioSystems Analyst AD instrument. A difference of 100 mP is considered to be an excellent response (FIG. 6).

### Example 4

### Erythropoietin Receptor Barnase Fusion Erythropoietin Binding Assay

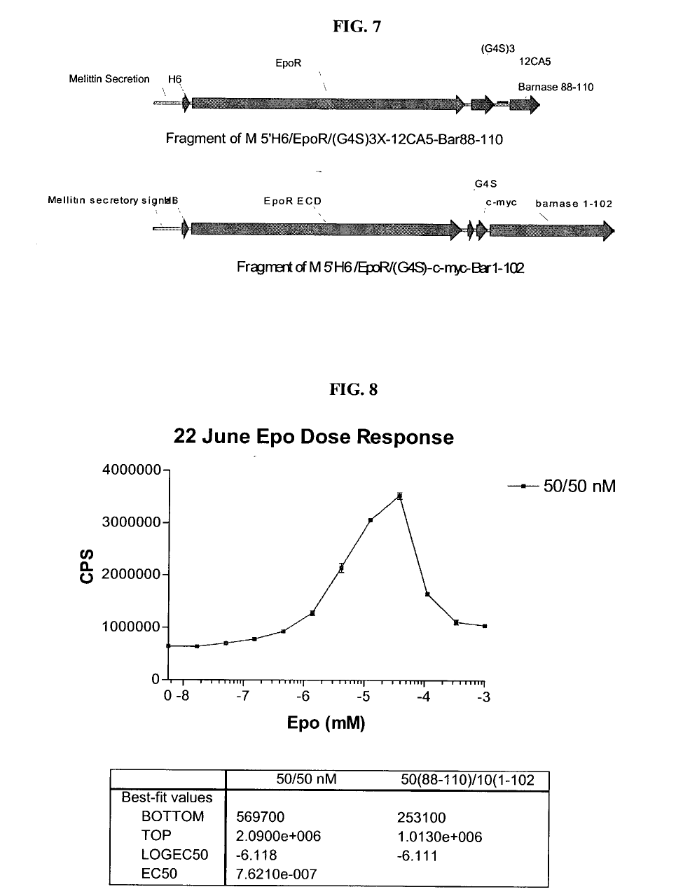

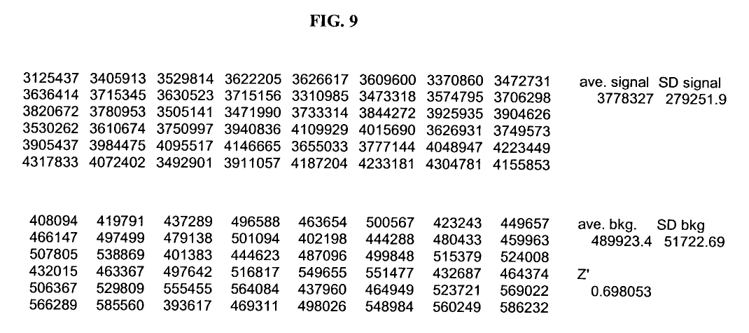

To evaluate the suitability of barnase fusions for high throughput screening, fusions of the extracellular domain of the erythropoietin receptor to the same complementing barnase domains as described in Example 2 were made (FIG. 7). Fusion proteins were expressed in insect cells and purified as described and a 50 nM solution of each EpoR fusion was used in the 96 well assay. The binding EC50 of 5 nM (FIG. 8) is equivalent to those obtained for Epo binding by Biazzo DE, Motamedi H, Mark DF, Qureshi SA. in “A high-throughput assay to identify compounds that can induce dimerization of the erythropoietin receptor.” (Anal Biochem Feb. 1, 2000 ;278(1):39-45). To characterize the value of the assay for high throughput screening 48 wells of a 96 well plate were assayed in the presence or absence of 50 nM Epo (FIG. 9). Z′ calculations were made as described in “A Simple Statistical Parameter for Use in Evaluation and Validation of High Throughput Screening Assays.” (Zhang JH, Chung TD, Oldenburg KR. J Biomol Screen 1999;4(2):67-73). The resulting value of 0.69 is significantly above the 0.5 threshold required for validation of a primary screening assay.

All patents, patent applications, and publications described herein are hereby incorporated by reference to the same extent as if each was specifically and individually indicated to be incorporated by reference.

The steps depicted and/or used in the methods herein may be performed in a different order than as depicted and/or stated. The steps are merely exemplary of the order these steps may occur. The steps may occur in any order that is desired, such that it still performs the goals of the claimed invention.

The inventions illustratively described herein can suitably be practiced in the absence of any element or elements, limitation or limitations, not specifically disclosed herein. Thus, for example, the terms “comprising”, “including,” “containing,” etc shall be read expansively and without limitation. Additionally, the terms and expressions contained herein have been used as terms of description and not of limitation, and there is no intention in the use of such terms and expression of excluding any equivalents of the future shown and described or portion thereof, but it is recognized that various modifications are possible within he scope of the invention claimed. Thus, it should be understood that although the present invention has been specifically disclosed by preferred embodiments and optional features, modification and variation of the inventions embodied therein herein disclosed can be resorted to by those skilled in the art, and that such modifications and variation are considered to be within the scope of the inventions disclosed herein. The inventions have described broadly and generically herein. Each of the narrower species and subgeneric groupings falling within the generic disclosure also form part of these inventions. This includes the generic description of each invention with a proviso or negative limitation removing and subject matter from the genus, regardless of whether or not the excised materials specifically resided herein. In addition, where features or aspects of an invention are described in terms of the Markush group, those schooled in the art will recognized that the invention is also thereby described in terms of any individual member or subgroup of members of the Markush group.

From the description of the invention herein, it is manifest that various equivalents can be used to implement the concepts of the present invention without departing from its scope. Moreover, while the invention has been described with specific reference to certain embodiments, a person or ordinary skill in he art would recognize that changes can be made in form and detail without departing from the spirit and the scope of the invention. The described embodiments are to be considered in all respects as illustrative and not restrictive. It should also be understood that the invention is not limited to the particular embodiments described herein, but is capable of many equivalents, rearrangements, modifications, and substitutions without departing from the scoped of the invention. Thus, additional embodiments are within the scope of the invention and within the following claims.

We claim:

1. A method for detecting binding between a first specific binding member and a second specific binding member, said method comprising:
providing a first binding member-complementing domain conjugate comprising said first specific binding member linked to a first complementing RNAse domain;
providing a second binding member-complementing domain conjugate comprising said second specific binding member linked to a second complementing RNAse domain;
combining in assay medium said first binding member-complementing domain conjugate, said second binding member-complementing domain conjugate, and a labeled probe;
determining the RNAse activity of said medium.

2. The method according to claim 1, wherein said labeled probe is a self-quenching fluorescence probe.

3. The method according to claim 1, wherein said labeled probe comprises an end bound to a solid support and a free end having an attached label.

4. The method according to claim 1, wherein the first complementing RNAse domain is barnase 1-102 or barnase 1-36.

5. The method according to claim 1, wherein the second complementing domain is barnase 88-110, barnase 95-110, or barnase 37-110.

6. The method according to claim 1, wherein the first and second complementing domains are derived from barnase polypeptide.

7. The method according to claim 6, wherein the first complementing RNAse domain is barnase 1-102 and the second complementing domain is barnase 88-110.

8. A method for evaluating the effect of an analyte on the ability of a first specific binding member to bind to a second specific binding member, said method comprising:
providing a first binding member-complementing domain conjugate comprising said first specific binding member linked to a first complementing RNAsc domain;
providing a second binding member-complementing domain conjugate comprising said second specific binding member linked to a second complementing RNAse domain;
combining in assay medium said analyte, said second binding member-complementing domain conjugate, said first binding member-complementing domain conjugate, and a labeled probe;
determining the RNAse activity of said medium.

9. The method according to claim 8, wherein said first binding member-complementing domain conjugate and said labeled probe are added to the medium after a sufficient incubation time for said analyte to bind to said second specific binding member.

10. The method according to claim 8, wherein said analyte, said second binding member-complementing domain conjugate, said first binding member-complementing domain conjugate, and said labeled probe are added concomitantly to said assay medium.

11. The method according to claim 8, wherein said second binding member-complementing domain conjugate and said label are combined in said medium before addition of said analyte and said first binding member-complementing domain conjugate.

12. The method according to claim 8, wherein said labeled probe is a self-quenching fluorescence probe.

13. The method according to claim 8, wherein said probe comprises an end bound to a solid support and a free end having an attached label.

14. The method according to claim 8, wherein the first complementing RNAse domain is barnase 1-102 or barnase 1-36.

15. The method according to claim 8, wherein the second complementing domain is barnase 88-110, barnase 95-110, or barnase 37-110.

16. The method according to claim 8, wherein the first and second complementing domains are derived from barnase polypeptide.

17. The method according to claim 16, wherein the first complementing RNAse domain is barnase 1-102 and the second complementing domain is barnase 88-110.
